(19) United States
(12) Patent Application Publication
(10) Pub. No.: US 2025/0049463 A1
(43) Pub. Date: Feb. 13, 2025

(54) MEDICAL INSTRUMENT WITH INTEGRAL POSITION SENSOR AND HALL EFFECT SENSOR

(71) Applicant: Acclarent, Inc., Irvine, CA (US)

(72) Inventors: Raymond Yue-sing TANG, Rosemead, CA (US); Gigi KOE, Anaheim, CA (US)

(21) Appl. No.: 18/798,709

(22) Filed: Aug. 8, 2024

Related U.S. Application Data
(60) Provisional application No. 63/531,687, filed on Aug. 9, 2023.

(51) Int. Cl. A61B 17/32; A61B 17/00; A61B 90/00

(52) U.S. Cl. CPC A61B 17/32002; A61B 90/08; A61B 2017/00039; A61B 2017/00199; A61B 2017/00398; A61B 2017/00876; A61B 2017/320032; A61B 2090/0811; A61B 2217/005

(57) ABSTRACT

An apparatus includes a body, a rotary member, a navigation sensor, and an alignment system. The rotary member is sized and configured to fit in an anatomical passageway of a patient; and is configured to rotate relative to the body about a rotational axis. The navigation sensor is configured to generate first signals indicative of a position of the body in three-dimensional space. The alignment system includes a magnet and a Hall effect sensor. The magnet is fixedly secured to the rotary member; and is configured to generate a magnetic field. The Hall effect sensor is fixedly secured to the body and is configured to detect a magnitude of the magnetic field and to generate second signals indicative of an angular position of the rotary member relative to the body about the rotational axis.

### CROSS-REFERENCE TO RELATED APPLICATIONS

This application claims the benefit of U.S. Provisional Application No. 63/531,687, filed Aug. 9, 2023, the entirety of which is incorporated herein by reference.

### BACKGROUND

Image-guided surgery (IGS) is a technique where a computer is used to obtain a real-time correlation of the location of an instrument that has been inserted into a patient's body to a set of preoperatively obtained images (e.g., a CT or MRI scan, 3-D map, etc.), such that the computer system may superimpose the current location of the instrument on the preoperatively obtained images. An example of an electromagnetic IGS navigation system that may be used in IGS procedures is the CARTO® 3 System by Biosense-Webster, Inc., of Irvine, California. In some IGS procedures, a digital tomographic scan (e.g., CT or MRI, 3-D map, etc.) of the operative field is obtained prior to surgery. A specially programmed computer is then used to convert the digital tomographic scan data into a digital map. During surgery, special instruments having sensors (e.g., electromagnetic coils that emit electromagnetic fields and/or are responsive to externally generated electromagnetic fields) are used to perform the procedure while the sensors send data to the computer indicating the current position of each surgical instrument. The computer correlates the data it receives from the sensors with the digital map that was created from the preoperative tomographic scan. The tomographic scan images are displayed on a video monitor along with an indicator (e.g., crosshairs or an illuminated dot, etc.) showing the real-time position of each surgical instrument relative to the anatomical structures shown in the scan images. The surgeon is thus able to know the precise position of each sensor-equipped instrument by viewing the video monitor even if the surgeon is unable to directly visualize the instrument itself at its current location within the body.

Surgical cutting instruments configured for removal of lesions, polyps and fibroids within the nasal cavity are known. Some configurations may include an elongated inner member rotatably coaxially disposed within a tubular outer member. The distal end of the outer member includes an opening, and the distal end of the inner member includes a cutting edge. The proximal ends of the two members may be connected to a handle directly or via a detachable hub. The inner member may be hollow and in communication with an aspiration port so that severed tissue, etc. can be aspirated out through the inner member. The cutting edge of the inner member may cooperate with an edge at the opening of the outer member to shear tissue. Such shearing edges may have any various configurations suitable for the particular type of tissue, such as bone tissue, mucosa, etc. To use such surgical cutting instrument, the distal end of the instrument is advanced to the target surgical site, and the opening positioned adjacent the tissue to be removed. The opening may be repositioned to address tissue that could not be accessed with the instrument in the previous position.

It may be desirable to provide enhanced real-time positional information about one or more components of a surgical cutting instrument. While several different surgical instruments and methods of use have been made for tissue removal within the nasal cavity, it is believed that no one prior to the inventors has made or used the inventions described in the appended claims.

### BRIEF DESCRIPTION OF THE DRAWINGS

The accompanying drawings, which are incorporated in and constitute a part of this specification, illustrate embodiments of the inventions, and, together with the general description of the inventions given above, and the detailed description of the embodiments given below, serve to explain the principles of the present inventions.

FIG. 1 depicts a schematic view of an example of a surgery navigation system being used on a patient seated in an example of a medical procedure chair, according to some embodiments.

FIG. 2 depicts a perspective view of an example of a tissue shaving instrument having a handle assembly, a shaft assembly, and a shaft assembly alignment system, according to some embodiments.

FIG. 3 depicts an exploded perspective fragmentary view of the shaft assembly of FIG. 2 having an outer tube and an inner cutting member, according to some embodiments.

FIG. 4 depicts a perspective view of the outer tube of FIG. 3, showing a flexible navigation sensor assembly disposed along an outer cylindrical surface of the outer tube, according to some embodiments.

FIG. 5 depicts a partial cross-sectional top view of the tissue shaving instrument of FIG. 2, showing the shaft assembly alignment system including a Hall effect sensor secured to the handle assembly, and further including a magnetic base detectable by the Hall effect sensor and secured to the inner cutting member of the shaft assembly, according to some embodiments.

FIG. 6 depicts a perspective view of the shaft assembly alignment system of FIG. 5, according to some embodiments.

The drawings are not intended to be limiting in any way, and it is contemplated that various embodiments of the inventions may be carried out in a variety of other ways, including those not necessarily depicted in the drawings. The accompanying drawings incorporated in and forming a part of the specification illustrate several aspects of the present inventions, and together with the description serve to explain the principles of the inventions; it being understood, however, that these inventions are not limited to the precise arrangements shown.

### DETAILED DESCRIPTION

The following description of certain examples of the inventions should not be used to limit the scope of the present inventions. Other examples, features, aspects, embodiments, and advantages of the inventions will become apparent to those skilled in the art from the following description, which is by way of illustration, one of the best modes contemplated for carrying out the inventions. As will be realized, the inventions are capable of other different and obvious aspects, all without departing from the inventions. Accordingly, the drawings and descriptions should be regarded as illustrative in nature and not restrictive.

For clarity of disclosure, the terms “proximal” and “distal” are defined herein relative to a surgeon, or other operator, grasping a surgical device. The term “proximal” refers to the position of an element arranged closer to the surgeon, and the term “distal” refers to the position of an element arranged further away from the surgeon. Moreover, to the extent that spatial terms such as “top,” “bottom,” “upper,” “lower,” “vertical,” “horizontal,” or the like are used herein with reference to the drawings, it will be appreciated that such terms are used for exemplary description purposes only and are not intended to be limiting or absolute. In that regard, it will be understood that surgical instruments such as those disclosed herein may be used in a variety of orientations and positions not limited to those shown and described herein.

Furthermore, the terms “about,” “approximately,” and the like as used herein in connection with any numerical values or ranges of values are intended to encompass the exact value(s) referenced as well as a suitable tolerance that enables the referenced feature or combination of features to function for the intended purpose described herein.

### I. EXAMPLE OF AN IMAGE GUIDED SURGERY NAVIGATION SYSTEM

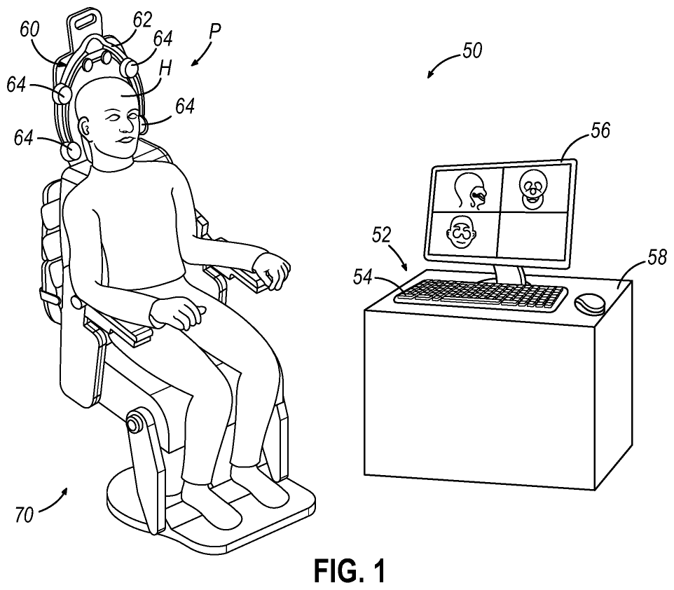

When performing a medical procedure within a head of a patient (P), it may be desirable to have information regarding the position of an instrument within the head (H) of the patient (P), particularly when the instrument is in a location where it is difficult or impossible to obtain an endoscopic view of a working element of the instrument within the head of the patient (P). FIG. 1 shows an example of an IGS navigation system 50 enabling a medical procedure to be performed within a head (H) of a patient (P) using image guidance. In addition to or in lieu of having the components and operability described herein the IGS navigation system 50 may be constructed and operable in accordance with at least some of the teachings of U.S. Pat. No. 7,720,521, entitled “Methods and Devices for Performing Procedures within the Ear, Nose, Throat and Paranasal Sinuses,” issued May 18, 2010, the disclosure of which is incorporated by reference herein, in its entirety; and/or U.S. Pat. No. 11,065,061, entitled “Systems and Methods for Performing Image Guided Procedures within the Ear, Nose, Throat and Paranasal Sinuses,” issued Jul. 20, 2021. The disclosures of U.S. Pat. No. 7,720,521, entitled “Methods and Devices for Performing Procedures within the Ear, Nose, Throat and Paranasal Sinuses,” issued May 18, 2010, the disclosure of which is incorporated by reference herein, in its entirety, and U.S. Pat. No. 11,065,061, entitled “Systems and Methods for Performing Image Guided Procedures within the Ear, Nose, Throat and Paranasal Sinuses,” issued Jul. 20, 2021, are incorporated by reference herein, in their entirety.

The IGS navigation system 50 of the present example comprises a field generator assembly 60, which comprises a set of magnetic field generators 64 that are integrated into a horseshoe-shaped frame 62. The field generators 64 are operable to generate alternating magnetic fields of different frequencies around the head (H) of the patient (P). An instrument may be inserted into the head (H) of the patient (P). Such an instrument may include one or more position sensors as described in greater detail below. In the present example, the frame 62 is mounted to a chair 70, with the patient (P) being seated in the chair 70 such that the frame 62 is located adjacent to the head (H) of the patient (P). By way of example only, the chair 70 and/or the field generator assembly 60 may be configured and operable in accordance with at least some of the teachings of U.S. Pat. No. 10,561,370, entitled “Apparatus to Secure Field Generating Device to Chair,” issued Feb. 18, 2020. The disclosure of U.S. Pat. No. 10,561,370, entitled “Apparatus to Secure Field Generating Device to Chair,” issued Feb. 18, 2020, is incorporated by reference herein, in its entirety. In some other variations, the patient (P) lies on a table; and the field generator assembly 60 is positioned on or near the table.

The IGS navigation system 50 of the present example further comprises a processor 52, which controls the field generators 64 and other elements of the IGS navigation system 50. For instance, the processor 52 is operable to drive the field generators 64 to generate alternating electromagnetic fields; and process signals from the instrument to determine the location of a navigation sensor in the instrument within the head (H) of the patient (P). The processor 52 comprises a processing unit (e.g., a set of electronic circuits arranged to evaluate and execute software instructions using combinational logic circuitry or other similar circuitry) communicating with one or more memories. The processor 52 of the present example is mounted in a console 58, which comprises operating controls 54 that include a keypad and/or a pointing device such as a mouse or trackball. A physician uses the operating controls 54 to interact with the processor 52 while performing the surgical procedure.

While not shown, the instrument may include a navigation sensor that is responsive to positioning within the alternating magnetic fields generated by the field generators 64. A coupling unit (not shown) may be secured to the proximal end of the instrument and may be configured to provide communication of data and other signals between the console 58 and the instrument. The coupling unit may provide wired or wireless communication of data and other signals.

In some versions, the navigation sensor of the instrument may comprise at least one coil at or near the distal end of the instrument. When such a coil is positioned within an alternating electromagnetic field generated by the field generators 64, the alternating magnetic field may generate electrical current in the coil, and this electrical current may be communicated along the electrical conduit(s) in the instrument and further to the processor 52 via the coupling unit. This phenomenon may enable the IGS navigation system 50 to determine the location of the distal end of the instrument within a three-dimensional space (i.e., within the head (H) of the patient (P), etc.). To accomplish this, the processor 52 executes an algorithm to calculate location coordinates of the distal end of the instrument from the position related signals of the coil(s) in the instrument.

The processor 52 uses software stored in a memory of the processor 52 to calibrate and operate the IGS navigation system 50. Such operation includes driving the field generators 64, processing data from the instrument, processing data from the operating controls 54, and driving a display screen 56. In some implementations, operation may also include monitoring and enforcement of one or more safety features or functions of the IGS navigation system 50. The processor 52 is further operable to provide video in real time via the display screen 56, showing the position of the distal end of the instrument in relation to a video camera image of the patient's head (H), a CT scan image of the patient's head (H), and/or a computer-generated three-dimensional model of the anatomy within and adjacent to the patient's nasal cavity. The display screen 56 may display such images simultaneously and/or superimposed on each other during the surgical procedure. Such displayed images may also include graphical representations of instruments that are inserted in the patient's head (H), such that the operator may view the virtual rendering of the instrument at its actual location in real time. By way of example only, the display screen 56 may provide images in accordance with at least some of the teachings of U.S. Pat. No. 10,463,242, entitled “Guidewire Navigation for Sinuplasty,” issued Nov. 5, 2019. The disclosure of U.S. Pat. No. 10,463,242, entitled “Guidewire Navigation for Sinuplasty,” issued Nov. 5, 2019, is incorporated by reference herein, in its entirety. In the event that the operator is also using an endoscope, the endoscopic image may also be provided on the display screen 56.

The images provided through the display screen 56 may help guide the operator in maneuvering and otherwise manipulating instruments within the patient's head (H). It should also be understood that other components of a surgical instrument and other kinds of surgical instruments, as described below, may incorporate a navigation sensor like the navigation sensor described above.

### II. EXAMPLE OF A SURGICAL CUTTING INSTRUMENT HAVING AN ALIGNMENT SYSTEM WITH A HALL EFFECT SENSOR

In some instances, it may be desirable to determine the angular position of a first, rotary component of an instrument relative to another component of the instrument, such as a second component of the instrument which the first component is configured to rotate relative to, in addition to determining the location of the distal end of the instrument within a three-dimensional space. In this regard, surgical cutting instruments such as tissue shaving instruments, debriders, and drills may include a first component in the form of a rotary cutting element that is configured to rotate relative to a second component. It may be desirable to determine the angular position of the rotary cutting element, such as while the rotary cutting element is rotating relative to the second component in addition to while the rotary cutting element is stationary relative to the second component. For example, it may be desirable to determine the angular position of an inner cutting member of a tissue shaving instrument relative to an outer tube of the tissue shaving instrument in order to determine whether a cutting window opening of the inner cutting member is in an opened or closed state (i.e., whether the lateral opening of the inner cutting member is angularly aligned with the lateral opening of the outer tube). Maintaining the cutting window opening in a closed state during advancement of the tissue shaving instrument alongside nearby tissues to access the target tissue and/or during retraction of the tissue shaving instrument alongside such nearby tissues may effectively terminate any suction that might otherwise be applied through the cutting window opening; and may thereby inhibit inadvertently withdrawing such nearby tissues into the tissue shaving instrument. Thus, determining the state of the cutting window opening and/or actively positioning the cutting window opening to the closed state in order to insert or remove the tissue shaving instrument may provide greater comfort and enhanced outcomes for the patient.

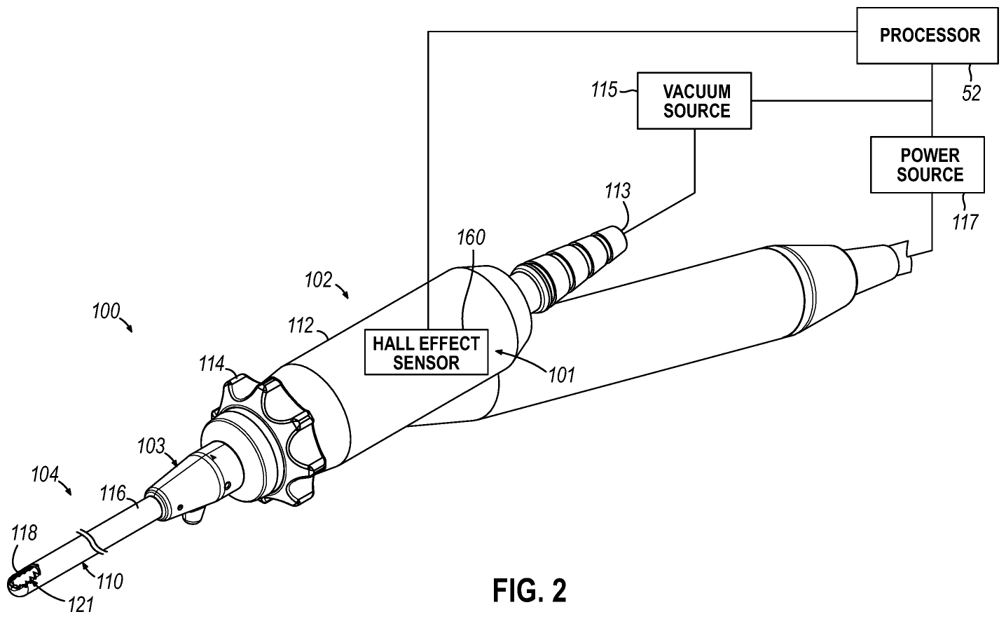

FIGS. 2-6 show an example of a surgical cutting instrument in the form of a tissue shaving instrument 100 including an alignment system 101 having such functionality. The tissue shaving instrument 100 may be used to sever and remove tissue, such as bone tissue and mucosal tissue, from an anatomical passageway. For instance, the tissue shaving instrument 100 may be used to sever and remove bone tissue and adjacent mucosal tissue from the nasal cavity, as well as from any other suitable location. The tissue shaving instrument 100 of this example also includes a handle assembly 102, a hub 103, a shaft assembly 104, and a navigation sensor assembly 110. While the alignment system 101 is shown and described in the context of the tissue shaving instrument 100, it will be appreciated that the alignment system 101 may be readily incorporated into any other suitable type of surgical instrument, such as any other type of surgical instrument having a rotary component configured to rotate relative to another component of the instrument. For example, the alignment system 101 may be readily incorporated into a debrider or a drill.

As shown in FIG. 2, the handle assembly 102 of this example includes a body 112 that is sized and configured to be grasped and operated by a single hand of an operator, such as via a power grip, a pencil grip, or any other suitable kind of grip. The handle assembly 102 may include controls for the operation of the instrument 100, or the controls may be located remotely. The instrument 100 further includes a suction port 113 which may be operatively connected to a suction source 115, which may be in operative communication with the processor 52, and which may be operable to selectively provide enough suction at a surgical site to pull severed tissue proximally through the instrument 100. In this manner, the suction port 113 may be configured to enable aspiration of tissue, such as a bone tissue, from a surgical site. Rotational motion may be delivered by a motorized drive assembly (not shown) within the handle assembly 102 to the shaft assembly 104, although any suitable rotational or oscillatory motion source may be utilized. For example, such motion source may be housed within the handle assembly 102 or may be external and connectable to the handle assembly 102. A power source 117, which may be in operative communication with the processor 52, may connect to the motorized drive assembly to power the instrument 100 for use. In addition, or alternatively, the handle assembly 102 may house a battery (not shown).

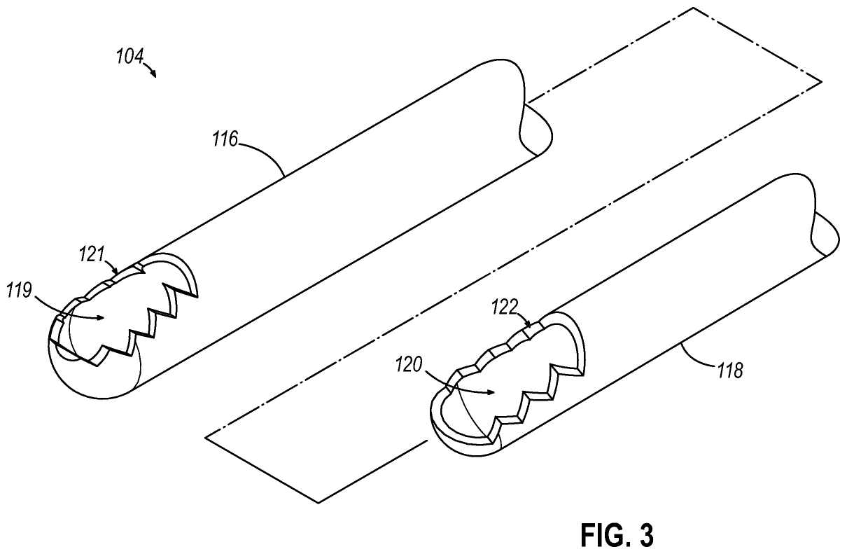

As best shown in FIG. 3, the shaft assembly 104 generally includes a longitudinally straight, rigid outer shaft 116 and an inner cutting member 118 collectively configured to receive and remove tissue from the surgical site. The cutting member 118, which may include a tube, is disposed within a longitudinally extending lumen 119 of the outer shaft 116 and is configured to be rotated about a longitudinal axis of the shaft assembly 104 at a distal portion. The cutting member 118 defines a suction lumen 120; and extends proximally to the handle assembly 112 and connects to the motorized drive assembly, which rotatably drives the cutting member 118 relative to the outer shaft 116. The outer shaft 116 includes a lateral shaft window opening 121 configured to cooperate with a cutting window opening 122 of the inner cutting member 118 to sever and remove tissue. By way of example, as the cutting member 118 moves in a clockwise direction, an edge of the lateral shaft window opening 121 provides an opposing surface to a cutting edge of the cutting window opening 122 whereby tissue may be severed through a shearing action to remove a cut tissue portion therefrom. The edges of the lateral shaft window opening 121 and the cutting window opening 122 may have any configuration which suitably cooperates with the other to sever tissue, such as a knife edge, a serrated edge, bipolar, monopolar or harmonic energy modality, or laser activated cutting edge.

The suction source 115 fluidly connects to the suction port 113 to draw a vacuum through the suction lumen 120 toward a proximal end 124 of the cutting member 118, while the motorized drive assembly rotatably drives the cutting member 118 within the outer shaft 116, such that the cutting member 118 rotates cyclically and repeatedly through an open state and a closed state in relation to the outer shaft 116. In the open state, the cutting window opening 122 at least partially angularly aligns with the shaft window opening 121 to fluidly communicate the vacuum throughout to the environment for receiving and suctioning tissue therein. In contrast, in the closed state, a tubular sidewall of the cutting member 118 angularly aligns with and covers the shaft window opening 121 to inhibit, and even terminate in some examples, further suctioning. Such a configuration may be configured and operable in accordance with any of the teachings of U.S. Pub. No. 2019/0388117, entitled “Surgical Shaver with Feature to Detect Window State,” published Dec. 26, 2019. The disclosure of U.S. Pub. No. 2019/0388117, entitled “Surgical Shaver with Feature to Detect Window State,” published Dec. 26, 2019, is incorporated by reference herein, in its entirety. As described in greater detail below, the shaft assembly alignment system 101 may be configured to effect movement of the cutting member 118 from the open state toward the closed state such that the cutting member 118 stops in the closed state to inhibit further suctioning of the tissue through the shaft window opening 121.

The shaft assembly 104 is also rotatable relative to the handle assembly 110, about the longitudinal axis of the shaft assembly 104. Such rotation may be driven via a rotation control knob 114, which is rotatably coupled with the body 112 of the handle assembly 110. Alternatively, the shaft assembly 104 may be rotated via some other form of user input; or may be non-rotatable relative to the handle assembly 110. It should also be understood that the example of the handle assembly 110 described herein is merely an illustrative example. The shaft assembly 104 may instead be coupled with any other suitable kind of handle assembly or other supporting body. In some cases, the shaft assembly 104 may be rotated about the longitudinal axis of the shaft assembly 104 via the control knob 114 to re-orient the angular position of the shaft window opening 121 about the longitudinal axis of the shaft assembly 104, to thereby promote access to a targeted tissue site.

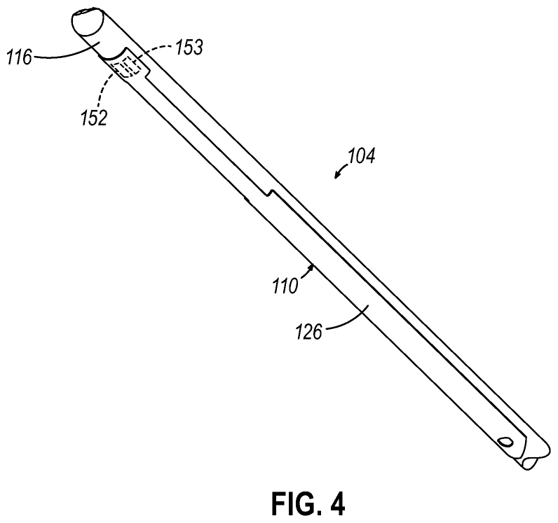

As best shown in FIG. 4, the navigation sensor assembly 110 is disposed on an exterior of the outer shaft 116 and is operable to provide navigation capabilities to the outer shaft 116. More particularly, the navigation sensor assembly 110 is disposed along a generally cylindrical outer surface of the outer shaft 116 in a generally curved configuration in which the navigation sensor assembly 110 is curved about the longitudinal axis of the outer shaft 116 with a radius of curvature corresponding to that of the cylindrical outer surface of the outer shaft 116 to thereby conform to an outer circumference of the outer shaft 116. In some versions, the instrument 100 may include a sheath (not shown) positioned coaxially about at least a portion of the outer shaft 116 radially outwardly of the navigation sensor assembly 110, such that the navigation sensor assembly 110 may be sandwiched between the sheath and the outer shaft 116.

The navigation sensor assembly 110 of this example is provided in the form of a flexible printed circuit board (PCB) and includes an elongate, generally rectangular flex circuit substrate 126 and a pair of distal navigation sensors 152, 153 that are operable to generate signals indicative of the position of the respective navigation sensor 152, 153 and thereby indicative of the position of at least a portion (e.g., the outer shaft 116) of the instrument 100 in three-dimensional space, when positioned within an alternating electromagnetic field generated by the field generators 64. The position data generated by such position related signals may be processed by the processor 52 for providing a visual indication to the operator to show the operator where the outer shaft 116 of the instrument 100 is located within the patient (P) in real time. Such a visual indication may be provided as an overlay on one or more preoperatively obtained images (e.g., CT scans) of the patient's anatomy. In the example shown, the distal navigation sensors 152, 153 are positioned at or near the lateral shaft window opening 121 of the outer shaft 116 for facilitating navigation of the lateral shaft window opening 121. The navigation sensor assembly 110 of the present version is disposed along a generally cylindrical outer surface of the outer shaft 116, such that one distal navigation sensor 152, 153 may be disposed on a first lateral side of the outer shaft 116 and the other distal navigation sensor 152, 153 may be disposed on a second lateral side of the outer shaft 116. In this manner, the pair of distal navigation sensors 152, 153 may provide position related signals indicative of locations of both lateral sides of the outer shaft 116, which may improve the accuracy of the location coordinates calculated by the processor 52. By way of example only, the navigation sensor assembly 110 may be configured and operable in accordance with any of the teachings of U.S. Pub. No. 2022/0257093, entitled “Flexible Sensor Assembly for ENT Instrument,” published Aug. 18, 2022. The disclosure of U.S. Pub. No. 2022/0257093, entitled “Flexible Sensor Assembly for ENT Instrument,” published Aug. 18, 2022, is incorporated by reference herein, in its entirety.

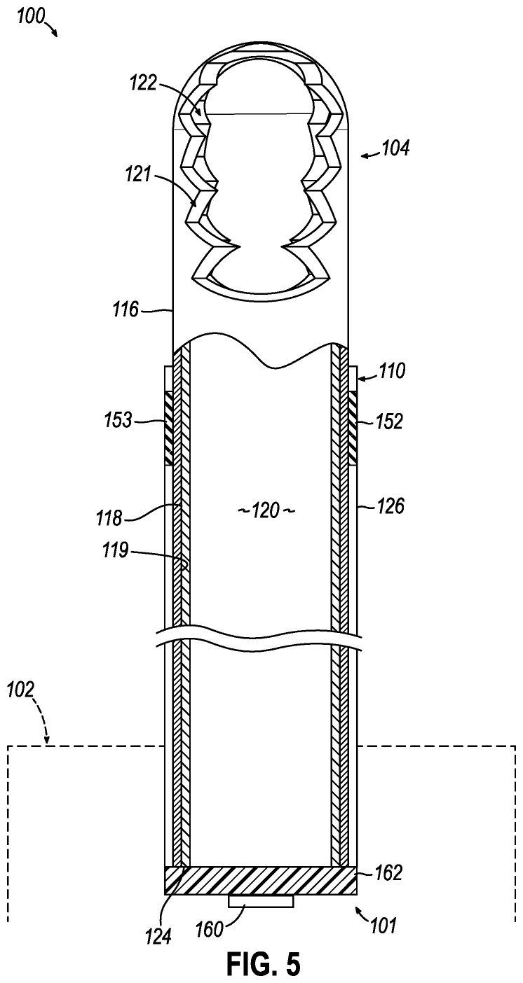

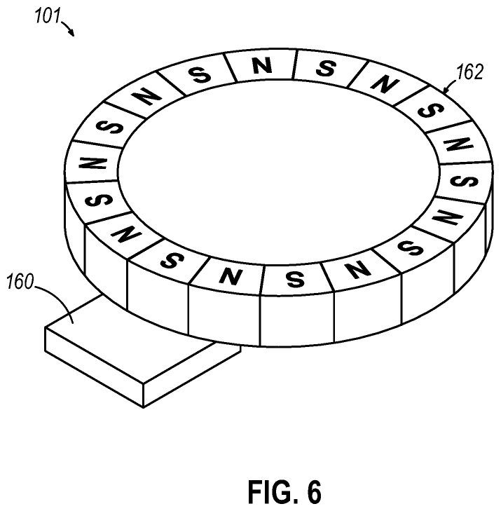

As noted above, the shaft assembly alignment system 101 may be configured to stop the cutting member 118 relative to the outer shaft 116 in the closed state to terminate suction through the shaft window opening 121 to inhibit inadvertently damaging tissue. In the present example, and as best shown in FIGS. 5 and 6, the shaft assembly alignment system 101 includes a Hall effect sensor 160 in operative communication with the processor 52 and fixedly secured to a portion of the handle assembly 102, such as the body 112, and a magnetic base 162 fixedly secured to the cutting member 118 at or near the proximal end 124 such that the magnetic base 162 is coaxial with the cutting member 118. In this manner, the magnetic base 162 is configured to rotate together with the cutting member 118 relative to the outer shaft 116, while the Hall effect sensor 160 may be configured to remain stationary relative to the outer shaft 116 during such rotation of the cutting member 118 relative to the outer shaft 116 (e.g., while the rotation control knob 114 is stationary). Thus, the angular position of the cutting window opening 122 is fixed relative to the magnetic base 162, while the angular position of the shaft window opening 121 may be fixed relative to the Hall effect sensor 160. In some other versions, the Hall effect sensor 160 may be fixedly secured directly to the outer shaft 116. The Hall effect sensor 160 is configured to detect the angular position of the magnetic base 162 and thereby detect the angular position of the cutting member 118 relative to the outer shaft 116 for determining when the cutting member 118 is in the closed state. In other words, the shaft assembly alignment system 101 is operable to generate signals indicating the angular position of the cutting window opening 122 relative to the shaft window opening 121. It be further understood that this data obtained through the shaft assembly alignment system 101 may be utilized in conjunction with data from the navigation sensor assembly 110 to determine the real-time position and orientation of the cutting window opening 122 in three-dimensional space.

The magnetic base 162 of the present example is generally disc-shaped, and includes a plurality of north and south pole magnets (N, S) arranged in a circumferentially-alternating manner and configured to generate a magnetic field, whose presence and magnitude are detectable by the Hall effect sensor 160 using the Hall effect. In some versions, the magnetic base 162 may include an opening (not shown) for allowing the suction lumen 120 to be fluidly connected to the suction port 113. For example, the magnetic base 162 may be generally annular.

The magnetic base 162 is configured and/or positioned relative to the Hall effect sensor 160 such that the magnitude of the magnetic field detected by the Hall effect sensor 160 varies based on the angular position of the magnetic base 162. For example, the Hall effect sensor 160 may be offset from a central axis of the magnetic base 162, such as by being positioned near an outer periphery of the magnetic base 162 as shown in FIG. 6. The Hall effect sensor 160 is operable to generate signals indicative of the angular position of the magnetic base 162 and thereby indicative of the angular position of the cutting member 118 relative to the outer shaft 116 based on the magnitude of the magnetic field detected by the Hall effect sensor 160. The angular position data generated by such angular position related signals may be processed by the processor 52 for determining the alignment of the cutting window opening 122 relative to the shaft window opening 121 and thus whether the cutting member 118 is in the open or closed state; and for providing a visual indication to the operator to indicate the open or closed state of the cutting member 118 in real time.

In the present example, the closed state occurs when the tubular sidewall of the cutting member 118 completely covers the shaft window opening 121 to terminate suction therethrough. The open state occurs when any portion of the cutting window opening 122 aligns with the shaft window opening 121 to communicate suction therethrough. In this respect, such open and closed states may each include a variety of positions for the cutting member 118 and the outer shaft 116, and the inventions are not intended to be unnecessarily limited to such states being single, discrete positions.

In use, the processor 52 may continuously monitor whether the cutting window opening 122 is in the open or closed state, and may direct the motorized drive assembly (e.g., via the power source 117) to drive movement of the cutting member 118. With each cyclical rotation of the cutting member 118, the Hall effect sensor 160 may detect the angular position of the magnetic base 162 and communicate such angular positions to the processor 52. The processor 52 may allow the motorized drive assembly to continue rotating the cutting member 118 so long as the operator selectively manipulates an activation control (not shown) to power the motorized drive assembly.

After the operator manipulates the activation control to selectively cease powering the motorized drive assembly, the processor 52 may direct the motorized drive assembly to transition the cutting member 118 from the open state to the closed state based on the detected angular position of the magnetic base 162. In one example, such transitioning by the motorized drive assembly is more particularly arresting movement of the cutting member 118 to stop the cutting member 118 in the closed state. In another example, such transitioning by the motorized drive assembly is more particularly powered driven movement of the cutting member 118 to stop the cutting member 118 in the closed state. Of course, any combination of arrested and driven movement of the cutting member 118 by the motorized drive assembly may be directed by the processor 52 such that the inventions are not intended to be unnecessarily limited to only arresting or driven movement of the cutting member 118.

In addition, or alternatively, the processor 52 may simply provide the operator with a visual indication of the angular position of the cutting member 118 relative to the outer shaft 116, and/or a visual indication of whether the cutting member 118 is in the open or closed state, such as while the cutting member 118 is stationary relative to the outer shaft 116. For example, the processor 52 need not necessarily direct the motorized drive assembly based on the detected angular position of the magnetic base 162. Rather, the processor 52 may allow the operator to observe the visual indication of the angular position of the cutting member 118 relative to the outer shaft 116, and/or a visual indication of whether the cutting member 118 is in the open or closed state; and allow the operator to decide whether to manipulate the activation control to either power or case powering the motorized drive assembly according to the operator's own preference.

Unlike other types of sensors, such as optical encoders, the ability of the Hall effect sensor 160 to detect the magnetic field generated by the magnetic base 162 for accurately determining the angular position of the magnetic base 162 may be unaffected by various environmental factors such as humidity. Moreover, the presence of particulates, debris, or other objects between the Hall effect sensor 160 and the magnetic base 162 may also have no effect on the ability of the Hall effect sensor 160 to detect the magnetic field, since the Hall effect sensor 160 does not depend upon a direct line of sight to the magnetic base 162. It will also be appreciated that the magnetic base 162 may enable monitoring the angular position of the cutting member 118 relative to the outer shaft 116 without requiring any electrical connection to the cutting member 118 (e.g., wires, slip rings, etc.), which might otherwise interfere with the ability of the cutting member 118 to rotate relative to the outer shaft 116 at desired frequencies.

### III. EXAMPLES OF COMBINATIONS

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

Example 1. An apparatus, comprising: (a) a body; (b) a rotary member sized and configured to fit in an anatomical passageway of a patient, the rotary member being configured to rotate relative to the body about a rotational axis; (c) a navigation sensor coupled to the body, the navigation sensor being configured to generate first signals indicative of a position of the body in three-dimensional space; and (d) an alignment system including: (i) a magnet fixedly secured to the rotary member, the magnet being configured to generate a magnetic field, and (ii) a Hall effect sensor fixedly secured to the body, the Hall effect sensor being configured to detect a magnitude of the magnetic field and to generate second signals indicative of an angular position of the rotary member relative to the body about the rotational axis.

Example 2. The apparatus of Example 1, the rotary member including a cutting member.

Example 3. The apparatus of any of Examples 1 through 2, the body including a handle body.

Example 4. The apparatus of any of Examples 1 through 3, the body including an outer shaft extending along the rotational axis and defining a lumen, the rotary member being rotatably disposed within the lumen.

Example 5. The apparatus of Example 4, the outer shaft including a shaft opening in fluid communication with an environment.

Example 6. The apparatus of Example 5, the rotary member including a cutting window opening configured to be at least partially angularly aligned with the shaft opening to define an open state, and configured to be fully angularly misaligned from the shaft opening to define a closed state.

Example 7. The apparatus of Example 6, the rotary member including a suction lumen extending along the rotational axis, the cutting window opening being in fluid communication with the suction lumen.

Example 8. The apparatus of any of Examples 6 through 7, further comprising a processor in operative communication with the Hall effect sensor to receive the second signals therefrom, the processor being configured to determine whether the cutting window opening is in the open state or the closed state based on the second signals.

Example 9. The apparatus of Example 8, the processor being configured to transition the cutting window toward the closed state.

Example 10. The apparatus of any of Examples 5 through 9, the navigation sensor being configured to generate first signals indicative of a position of the shaft opening in three-dimensional space.

Example 11. The apparatus of any of Examples 1 through 10, the navigation sensor being configured to generate first signals indicative of a position of a distal portion of the body in three-dimensional space.

Example 12. The apparatus of any of Examples 1 through 11, the magnet being fixedly secured to a proximal end of the rotary member.

Example 13. The apparatus of Example 12, further comprising a magnetic base centered on the rotational axis, the magnetic base including the magnet.

Example 14. The apparatus of Example 13, the magnet including a plurality of north and south pole magnets in a circumferentially-alternating arrangement.

Example 15. The apparatus of any of Examples 1 through 14, the Hall effect sensor being offset from the rotational axis.

Example 16. An apparatus, comprising: (a) a shaft extending along a longitudinal axis, the shaft including: (i) a lumen, and (ii) a shaft opening in fluid communication with an environment; (b) a cutting member disposed within the lumen of the shaft and configured to rotate relative to the shaft about the longitudinal axis between an open state and a closed state, the cutting member including: (i) a suction lumen extending along the longitudinal axis, and (ii) a cutting window opening in fluid communication with the suction lumen, the cutting window opening being configured to be at least partially aligned with the shaft opening to define the open state, and configured to be fully misaligned from the shaft opening to define the closed state; (c) a navigation sensor coupled to the shaft, the navigation sensor being configured to generate first signals indicative of a position of the shaft in three-dimensional space; and (d) an alignment system including: (i) a magnet fixedly secured to the cutting member, the magnet being configured to generate a magnetic field, and (ii) a Hall effect sensor secured against movement relative to the shaft, the Hall effect sensor being configured to detect a magnitude of the magnetic field and to generate second signals indicative of an angular position of the cutting member relative to the shaft about the longitudinal axis.

Example 17. The apparatus of Example 16, the navigation sensor being configured to generate first signals indicative of a position of the shaft opening in three-dimensional space.

Example 18. The apparatus of any of Examples 16 through 17, further comprising a magnetic base fixedly secured to a proximal end of the cutting member, the magnetic base including the magnet.

Example 19. The apparatus of Example 18, the magnetic base being centered on the longitudinal axis, the Hall effect sensor being offset from the longitudinal axis.

Example 20. A method of operating a surgical instrument, the method comprising: (a) rotating a rotary member of the surgical instrument relative to a body of the surgical instrument about a rotational axis within an anatomical passageway of a patient, the rotary member including a magnet configured to generate a magnetic field; (b) receiving a first signal from a navigation sensor coupled to the body; (c) determining a position of the body in three-dimensional space based on the first signal; (d) receiving a second signal from a Hall effect sensor fixedly secured to the body, the second signal being indicative of a detected magnitude of the magnetic field; and (e) determining an angular position of the rotary member relative to the body about the rotational axis based on the second signal.

### IV. MISCELLANEOUS

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present inventions, further adaptations of the methods and systems described herein may be accomplished by appropriate modifications by one of ordinary skill in the art without departing from the scope of the present inventions. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present inventions should be considered in terms of the following claims and is understood not to be limited to the details of structure and operation shown and described in the specification and drawings.

What is claimed is:

1. A medical instrument comprising:
a body;
a rotary member sized and configured to fit in an anatomical passageway of a patient, the rotary member being configured to rotate relative to the body about a rotational axis;
a navigation sensor coupled to the body, the navigation sensor being configured to generate first signals indicative of a position of the body in three-dimensional space; and
an alignment system comprising:
a magnet fixedly secured to the rotary member, the magnet being configured to generate a magnetic field, and
a Hall effect sensor fixedly secured to the body, the Hall effect sensor being configured to detect a magnitude of the magnetic field and to generate second signals indicative of an angular position of the rotary member relative to the body about the rotational axis.

2. The medical instrument of claim 1, wherein the rotary member comprises a cutting member.

3. The medical instrument of claim 1, wherein the body comprises a handle body.

4. The medical instrument of claim 1, wherein the body comprises an outer shaft extending along the rotational axis and defining a lumen, the rotary member being rotatably disposed within the lumen.

5. The medical instrument of claim 4, wherein the outer shaft comprises a shaft opening in fluid communication with an environment.

6. The medical instrument of claim 5, wherein the rotary member comprises a cutting window opening configured to be (a) at least partially angularly aligned with the shaft opening to define an open state and (b) fully angularly misaligned from the shaft opening to define a closed state.

7. The medical instrument of claim 6, wherein the rotary member comprises a suction lumen extending along the rotational axis, the cutting window opening being in fluid communication with the suction lumen.

8. The medical instrument of claim 6, further comprising a processor in operative communication with the Hall effect sensor to receive the second signals therefrom, the processor being configured to determine whether the cutting window opening is in the open state or the closed state based on the second signals.

9. The medical instrument of claim 8, wherein the processor is configured to transition the cutting window toward the closed state.

10. The medical instrument of claim 5, wherein the navigation sensor is configured to generate first signals indicative of a position of the shaft opening in three-dimensional space.

11. The medical instrument of claim 1, wherein the navigation sensor is configured to generate first signals indicative of a position of a distal portion of the body in three-dimensional space.

12. The medical instrument of claim 1, wherein the magnet is fixedly secured to a proximal end of the rotary member.

13. The medical instrument of claim 12, further comprising a magnetic base centered on the rotational axis, the magnetic base including the magnet.

14. The medical instrument of claim 13, wherein the magnet comprises a plurality of north and south pole magnets in a circumferentially-alternating arrangement.

15. The medical instrument of claim 1, wherein the Hall effect sensor is offset from the rotational axis.

16. A medical instrument comprising:
a shaft extending along a longitudinal axis, the shaft comprising:
a lumen, and
a shaft opening in fluid communication with an environment;
a cutting member disposed within the lumen of the shaft and configured to rotate relative to the shaft about the longitudinal axis between an open state and a closed state, the cutting member comprising:
a suction lumen extending along the longitudinal axis, and
a cutting window opening in fluid communication with the suction lumen, the cutting window opening being configured to be at least partially aligned with the shaft opening to define the open state, and configured to be fully misaligned from the shaft opening to define the closed state;
a navigation sensor coupled to the shaft, the navigation sensor being configured to generate first signals indicative of a position of the shaft in three-dimensional space; and
an alignment system comprising:
a magnet fixedly secured to the cutting member, the magnet being configured to generate a magnetic field, and
a Hall effect sensor secured against movement relative to the shaft, the Hall effect sensor being configured to detect a magnitude of the magnetic field and to generate second signals indicative of an angular position of the cutting member relative to the shaft about the longitudinal axis.

17. The medical instrument of claim 16, wherein the navigation sensor is configured to generate first signals indicative of a position of the shaft opening in three-dimensional space.

18. The medical instrument of claim 16, further comprising a magnetic base fixedly secured to a proximal end of the cutting member, the magnetic base comprising the magnet.

19. The medical instrument of claim 18, wherein the magnetic base is centered on the longitudinal axis, the Hall effect sensor being offset from the longitudinal axis.

20. A method of operating a surgical instrument, the method comprising:
rotating a rotary member of the surgical instrument relative to a body of the surgical instrument about a rotational axis within an anatomical passageway of a patient, the rotary member comprising magnet configured to generate a magnetic field;
receiving a first signal from navigation sensor coupled to the body;
determining a position of the body in three-dimensional space based on the first signal;
receiving a second signal from a Hall effect sensor fixedly secured to the body, the second signal being indicative of a detected magnitude of the magnetic field; and
determining an angular position of the rotary member relative to the body about the rotational axis based on the second signal.
